(19) United States
(12) Patent Application Publication
(10) Pub. No.: US 2019/0134362 A1
(43) Pub. Date: May 9, 2019

(54) DRAIN TUBE HOLDER SYSTEMS

(71) Applicant: Red Oak Innovations, LLC, Moorhead, MN (US)

(72) Inventors: Louan R. FEE, Moorhead, MN (US); Patrick FEE, Moorhead, MN (US)

(21) Appl. No.: 16/238,282

(22) Filed: Jan. 2, 2019

Related U.S. Application Data
(63) Continuation-in-part of application No. 14/842,924, filed on Sep. 2, 2015, now Pat. No. 10,207,079.

(51) Int. Cl. A61M 27/00

(52) U.S. Cl. CPC A61M 27/00; A61M 2209/088

(57) ABSTRACT

Drain tube holder systems for securely retaining drainage tubes and reservoir bulbs against the body without impeding movement of the wearer. The drain tube holder systems generally includes a strap adapted to be secured around various portions of an individual's body such as the waist, thigh, or shoulder. A retainer attachment is fixedly or removably secured to the strap. The retainer attachment includes one or more pouches adapted to retain one or more tubes and bulbs therein, with a first end of the tube exiting the body of the individual before being secured within a tube retainer within the pouch. The pouch is also adapted to securely retain a bulb, such as a reservoir, adapted to retain fluids drained through the tube. Using the drain tube holder systems described herein, an individual may enjoy improved comfort while wearing a drainage tube, such as after a medical procedure.

This application is a continuation-in-part of U.S. patent application Ser. No. 14/842,924, filed on Sep. 2, 2015, the entire contents of which are incorporated herein by reference.

### FIELD

This disclosure relates generally to a tube holder and more specifically to a drain tube holder system for securely retaining drainage tubes and reservoir bulbs against the body without impeding movement of the wearer.

### BACKGROUND

Any discussion of the related art throughout the specification should in no way be considered as an admission that such related art is widely known or forms part of common general knowledge in the field.

Drain tubes are commonly used in the medical field for such reasons as extracting blood, pus, or any other built-up fluids from a wound or preventing accumulations of air. The drain tubes are typically placed in a patient by a surgeon and are common to many different medical procedures. The drain tubes are often a source of major discomfort and hardship for the patient as well as the clinical staff. Despite having been used in the medical field for many years, a system has not yet been introduced to comfortably and effectively retain the drainage tubes against the body of an individual.

Because of the inherent problems with the related art, there is a need for a new and improved drain tube holder system for securely retaining drainage tubes and reservoir bulbs against the body without impeding movement of the wearer.

### SUMMARY

Provided herein is a tube holder which includes a strap adapted to be secured around various portions of an individual's body such as the waist, thigh, or shoulder. A retainer attachment is fixedly or removably secured to the strap. The retainer attachment includes one or more pouches adapted to retain one or more tubes and bulbs therein, with a first end of the tube exiting the body of the individual before being secured within a tube retainer within the pouch. The pouch is also adapted to securely retain a bulb, such as a reservoir, adapted to retain fluids drained through the tube.

There has thus been outlined, rather broadly, some of the features of the disclosed embodiments in order that the detailed description thereof may be better understood, and in order that the present contribution to the art may be better appreciated. There are additional features that will be described hereinafter and that may form the subject matter of the claims appended hereto. It is to be understood that the claims are not necessarily limited to the details of construction or to the arrangements of the components set forth in the following description or illustrated in the drawings. Also, it is to be understood that the phraseology and terminology employed herein are for the purpose of the description and should not be regarded as limiting.

### DRAWINGS

FIG. 1 is an upper perspective view of one embodiment of a drain tube holder system described herein being worn around the mid-section of an individual.

FIG. 2 is an upper perspective view of the drain tube holder system of FIG. 1.

FIG. 3 is an upper perspective view of the drain tube holder system of FIG. 2 with the retainer attachment partially removed from the strap.

FIG. 4 is a cutaway frontal view of the drain tube holder system of FIGS. 1-3.

FIG. 5 is a top view of the drain tube holder system of FIGS. 1-4.

FIG. 6 is a frontal sectional view of the drain tube holder system of FIGS. 1-5.

FIG. 7 is a top view of the drain tube holder system of FIGS. 1-6 with a plurality of tube retainers arranged into rows.

FIG. 8 is a frontal sectional view of the drain tube holder system of FIG. 7.

FIG. 9 is an upper perspective view of another embodiment of a drain tube holder system in use around the mid-section of an individual.

FIG. 10 is an upper perspective view of the drain tube holder system of FIG. 9 utilizing a ring structure to secure the strap to the retainer attachment.

FIG. 11 is an upper perspective view of the embodiment shown in FIG. 10 in a closed position.

FIG. 12 is an upper perspective view of a drain tube holder system similar to the embodiment in FIG. 9 being worn around the thigh of an individual.

FIG. 13 is an upper perspective view of the drain tube holder system of FIG. 12 with one pouch.

FIG. 14 is an upper perspective view of another embodiment of a drain tube holder system worn around the shoulder in a satchel configuration.

FIG. 15 is an upper perspective view of the drain tube holder system of FIG. 14.

FIG. 16 is an upper perspective view of a drain tube holder system in the form of a pouch being retained on a bandage by a pouch retainer.

FIG. 17 is a frontal perspective view of the pouch of FIG. 16.

FIG. 18 is a rear perspective view of the pouch of FIGS. 16 and 17.

FIG. 19 is an upper perspective view of another embodiment of a drain tube holder system in which a cloth divider is utilized to separate the two pouches.

FIG. 20 is an upper perspective view of another embodiment of a drain tube holder system that is similar to the embodiment of FIG. 2 but with the drain tube holders located above the pouches on the strap.

FIG. 21 is an upper perspective view of still another embodiment of a drain tube holder system that is similar to the embodiment of FIG. 2 but with the drain tube holders located above the pouches on the base of the retainer attachment.

FIG. 22 is a view similar to FIG. 18 showing alternative embodiments of pouch retainers and alternative pouch retainer locations.

FIG. 23 is an upper perspective view of another embodiment of a drain tube holder system where the pouch(es) face toward the individual.

FIG. 24 is a rear perspective view of the pouch(es) of FIG. 23.

### DETAILED DESCRIPTION

Overview

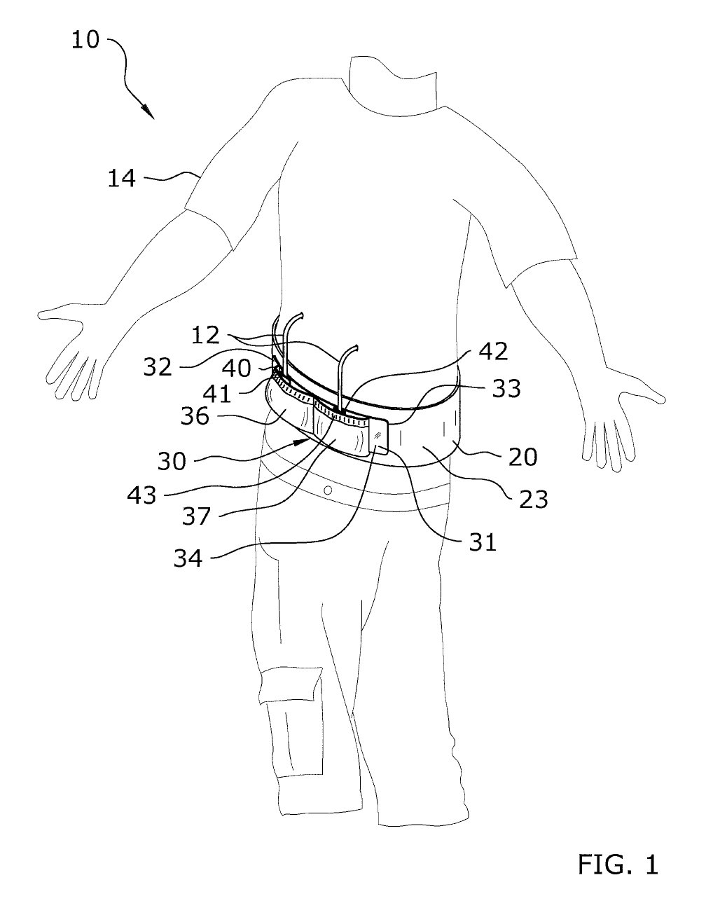

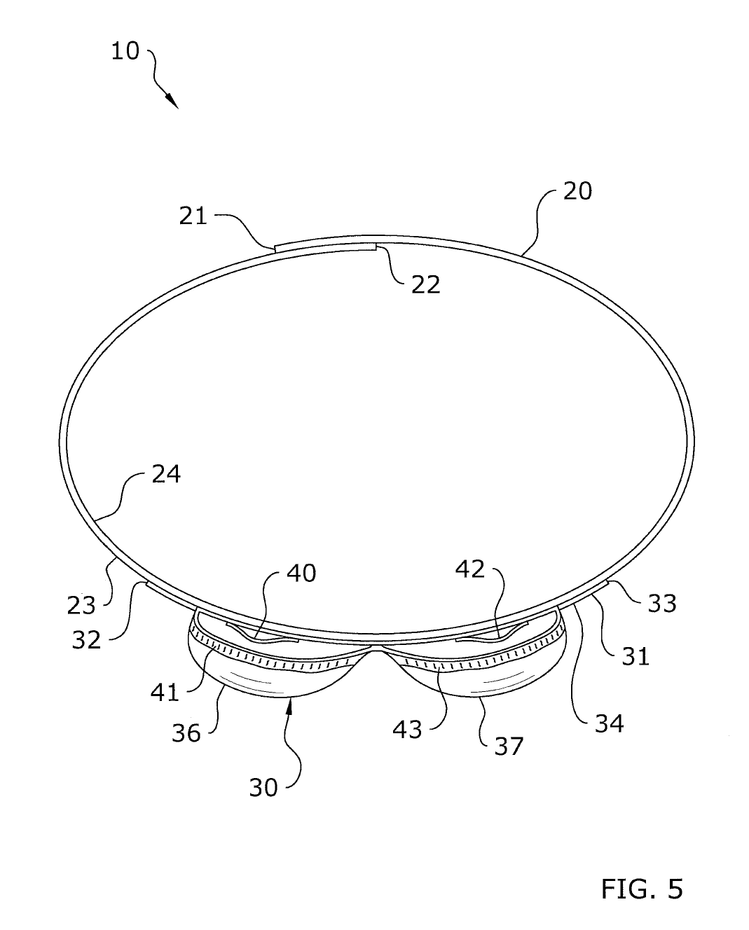

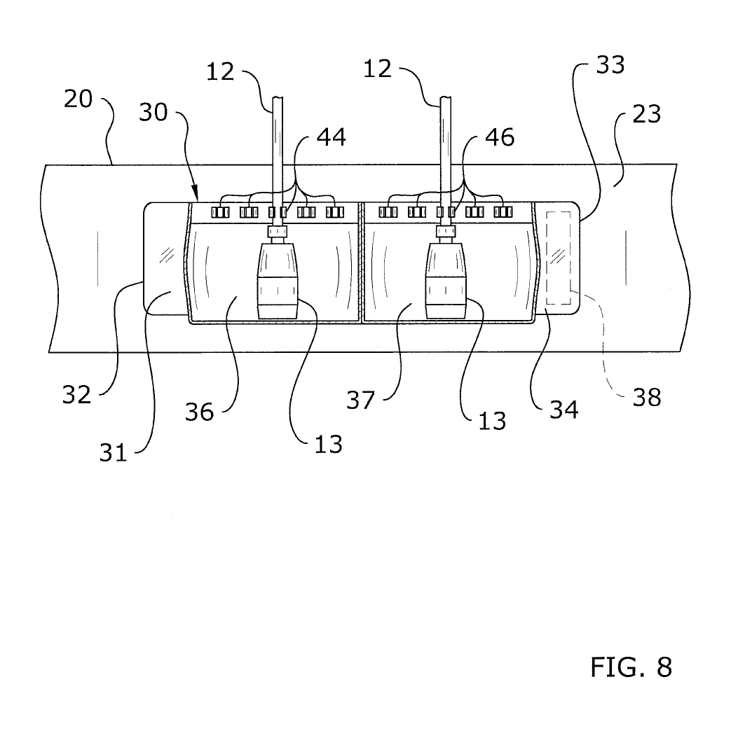

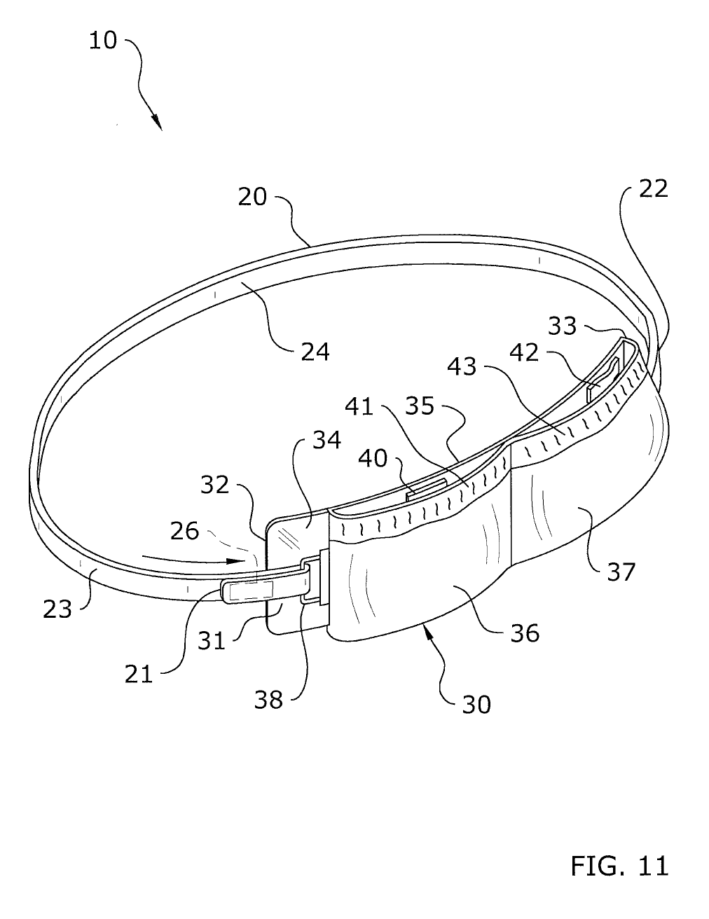

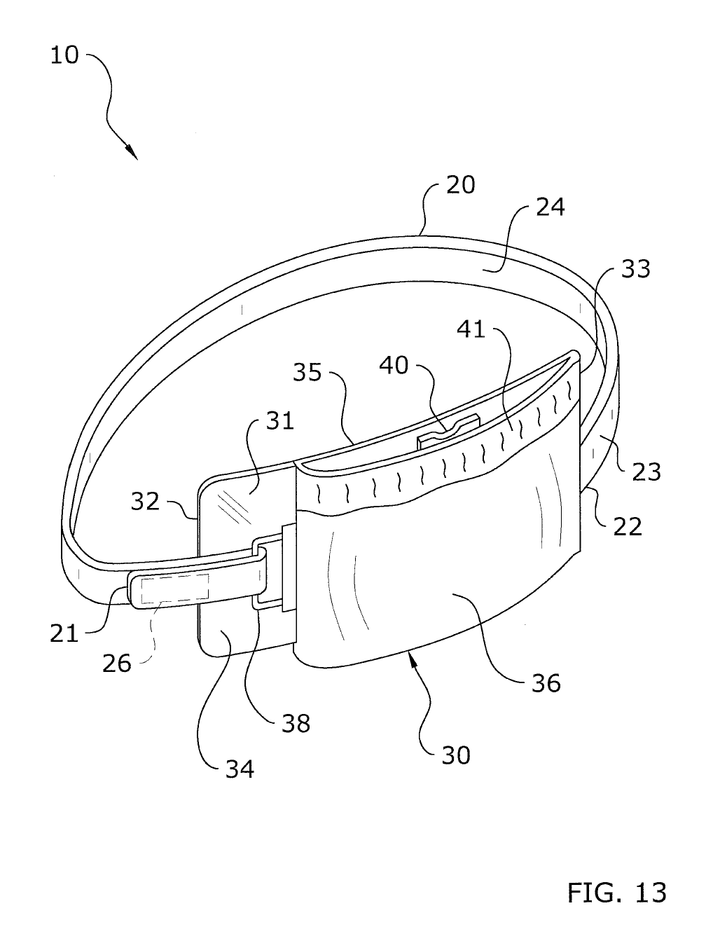

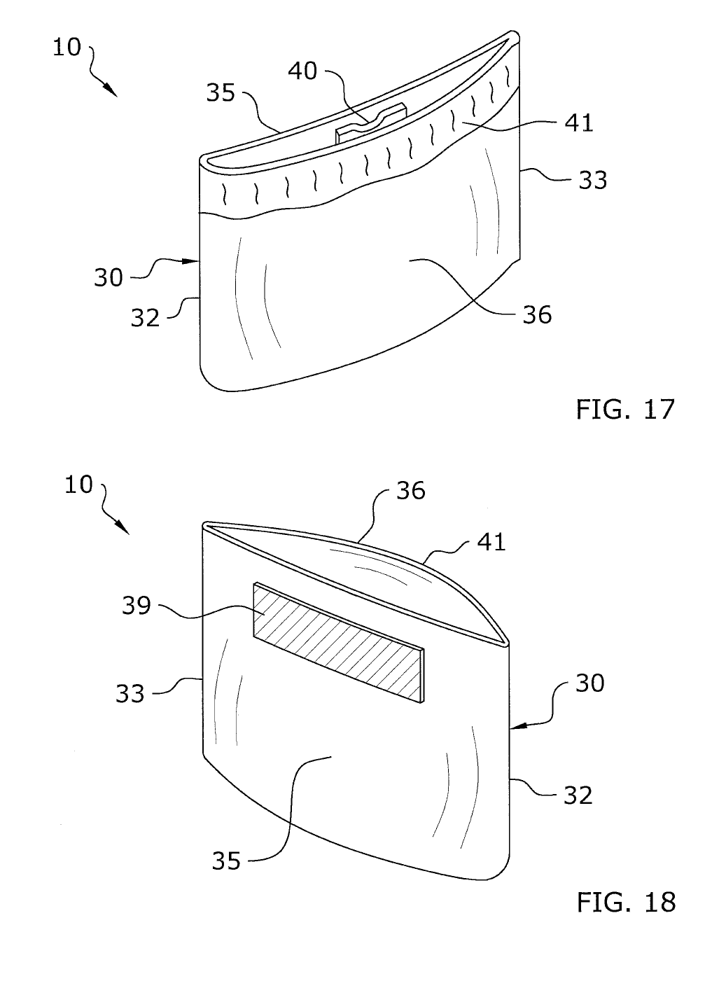

Turning now descriptively to the drawings, in which similar reference characters denote similar elements throughout the several views, FIGS. 1 through 19 illustrate a drain tube holder system 10, which comprises a strap 20 adapted to be secured around various portions of an individual's 14 body such as the waist, thigh, or shoulder. A retainer attachment 30 is fixedly or removably secured to the strap 20. The retainer attachment 30 includes one or more pouches 36, 37 adapted to retain one or more tubes 12 and bulbs 13 therein, with a first end of the tube 12 exiting the body of the individual 14 before being secured within a tube retainer 40, 42 within the pouch 36, 37. The pouch 36, 37 is also adapted to securely retain a bulb 13, such as a reservoir, adapted to retain fluids drained through the tube 12.

Strap

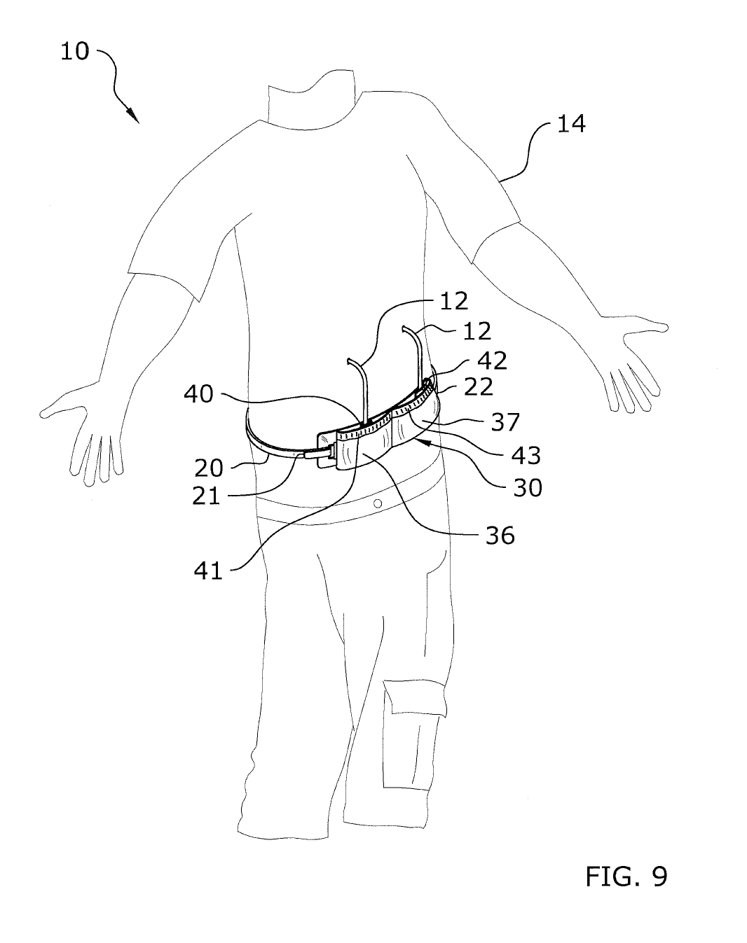

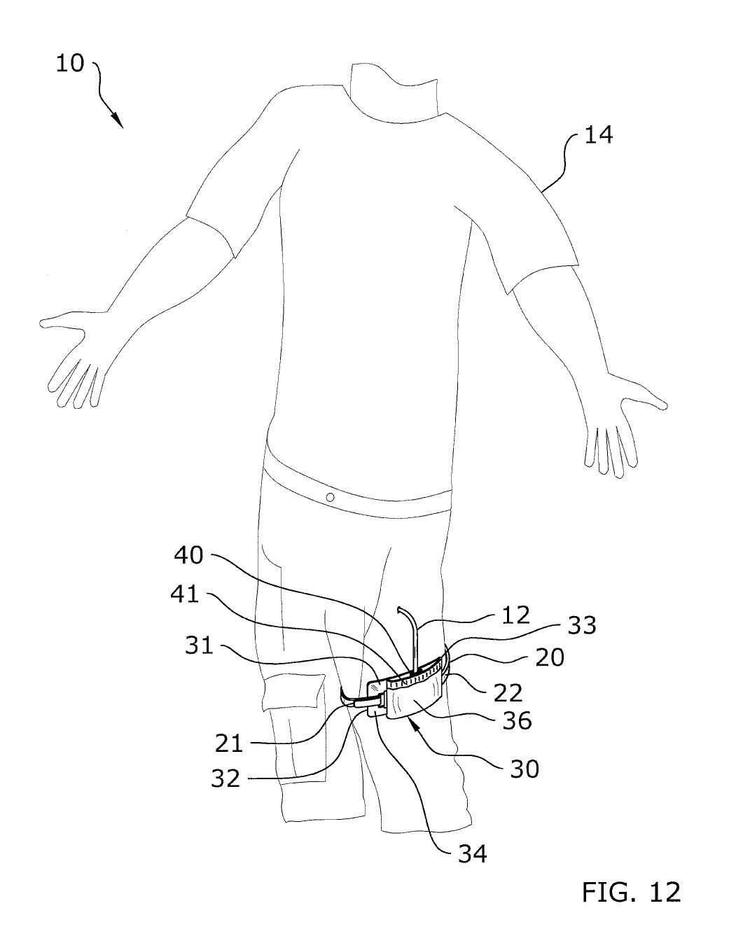

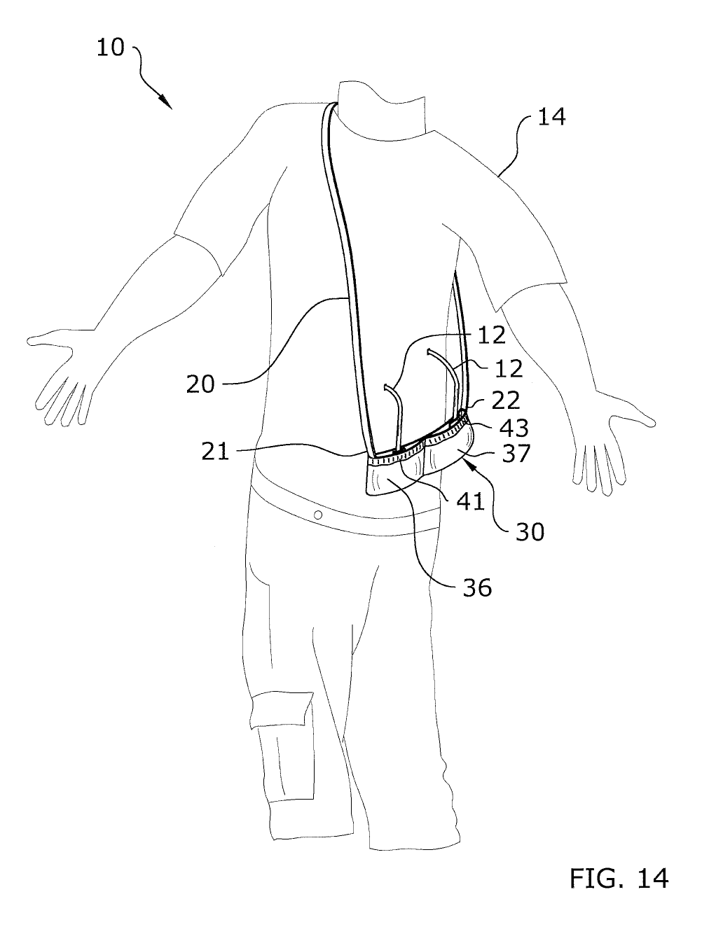

As shown throughout the figures, some of the illustrated embodiments utilize at least one strap 20 for securing the disclosed embodiments to the body of an individual 14. FIGS. 1 and 9 illustrate a waist strap 20 adapted to wrap around the waist like a belt. This embodiment shows the strap 20 as comprising an abdominal compression binder with a retainer attachment. FIG. 12 illustrates the strap 20 being secured around the leg of an individual 14. FIG. 14 illustrates a shoulder strap 20 adapted to be secured around the body of the individual 14 similar to a satchel.

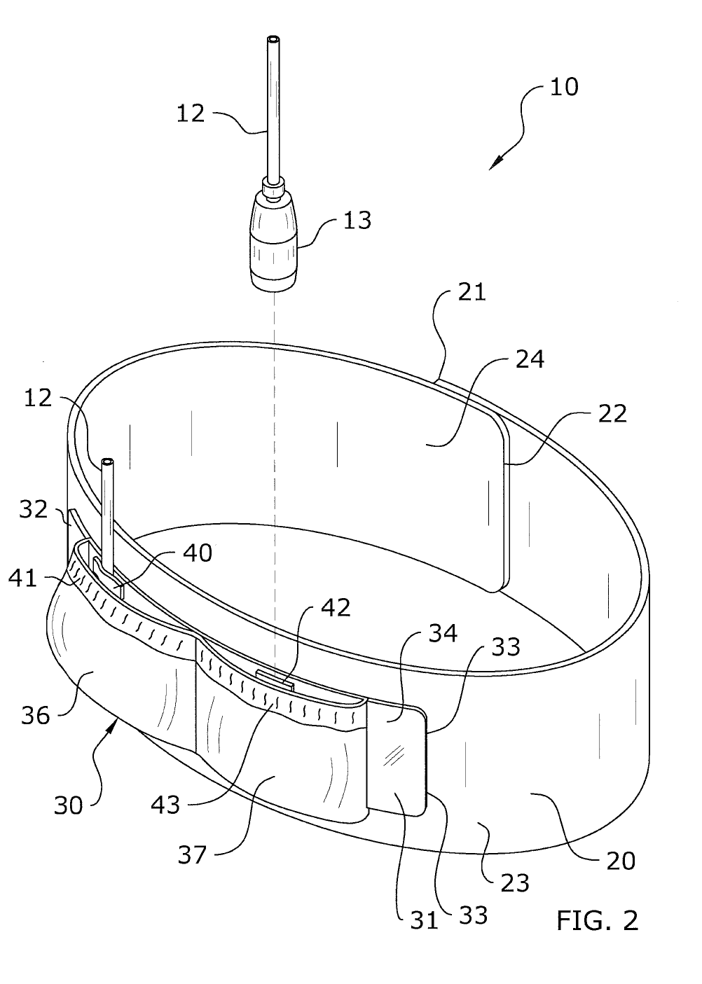

As shown in FIG. 2, the strap 20 generally comprises a first end 21 and a second end 22, with the first and second ends 21, 22 being adapted to secure to each other to form a continuous loop which may be placed around various portions of the body, such as the shoulder, waist, or leg as shown in the figures. The configuration, size, and orientation of the strap 20 may vary in different embodiments. The strap 20 will preferably be sized to comfortably fit around the limb or other body portion of the individual 14 who is wearing it.

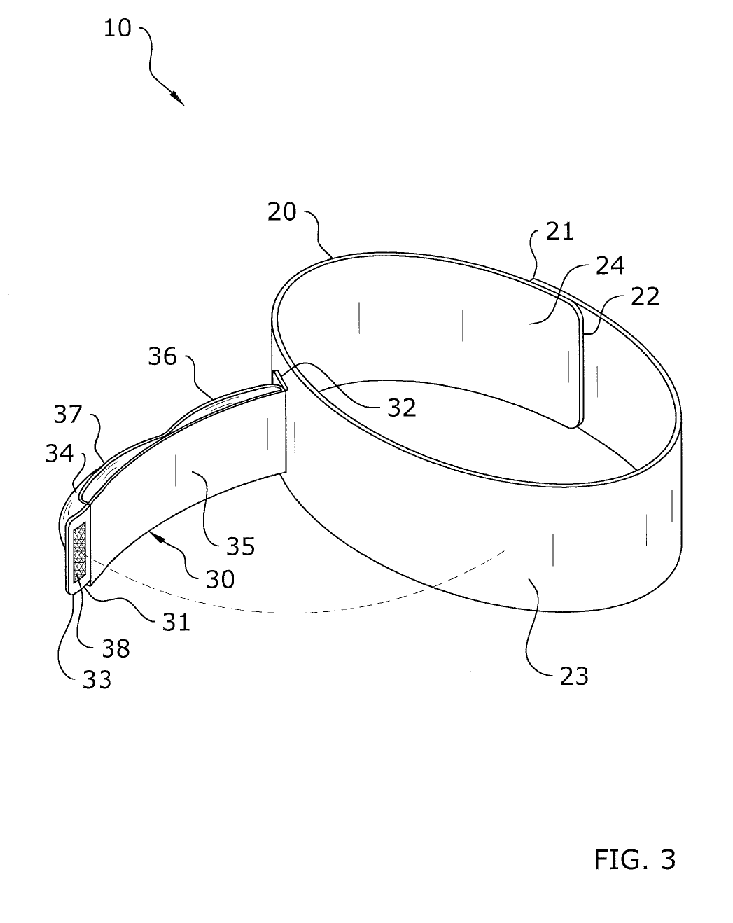

As shown in FIG. 2, the strap 20 includes an outer surface 23 which faces away from the body and an inner surface 24 which faces toward the body. A retainer attachment 30, described below, may be fixedly or removably attached to the outer surface 23 of the strap 20, such as shown in FIG. 3. While the strap 20 may comprise various materials and should not be limited to any particular material, the outer surface 23 of the strap 20 may comprise a material adapted to removably engage with a hook-and-loop fastener such as Velcro.

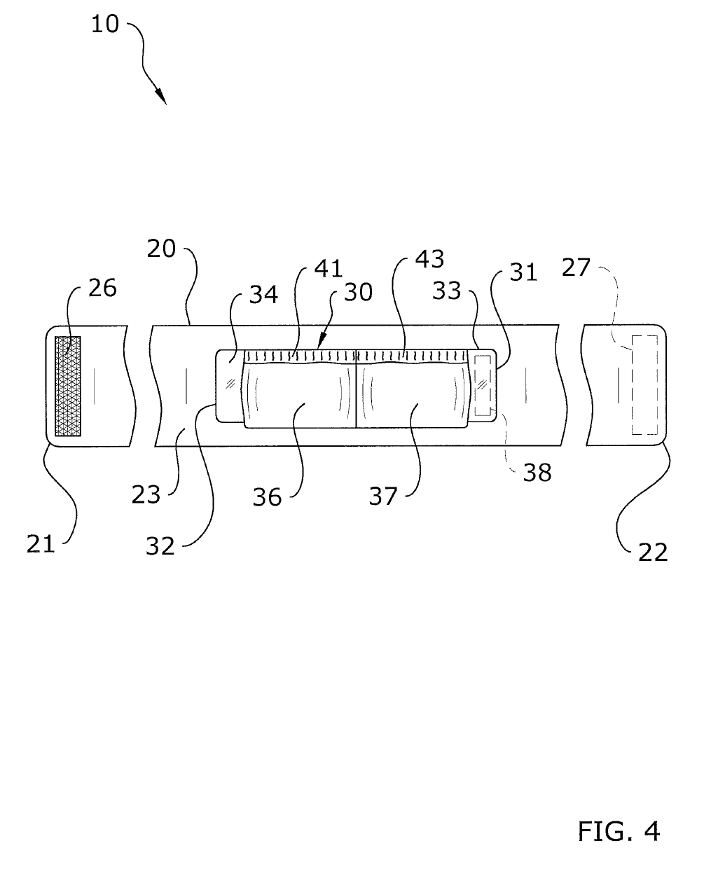

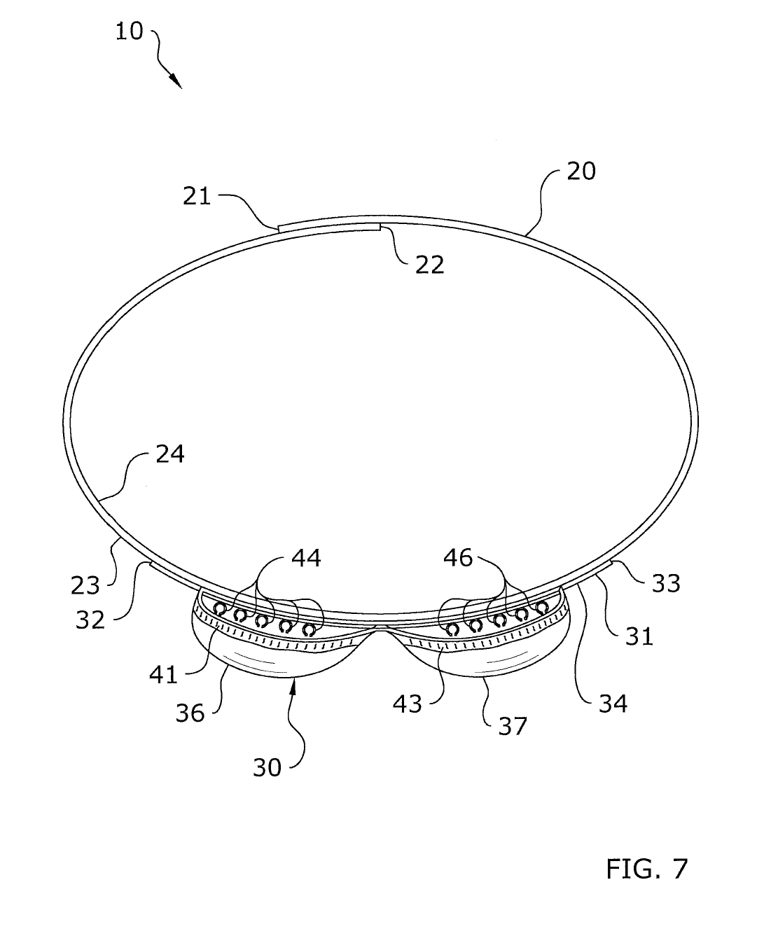

The strap 20 will generally include at least one connector 26, 27 for securing the first and second ends 21, 22 of the strap 20 together to form a loop. In a preferred embodiment as shown in the figures, a first connector 26 and a second connector 27 are utilized. For example, as shown in FIG. 4, a first connector 26 may be positioned adjacent to the first end 21 of the strap 20 on its outer surface 23. A corresponding second connector 27 may be positioned adjacent to the second end 22 of the strap 20 on its inner surface 24. Thus, the first and second connectors 26, 27, illustrated as hook-and-loop fasteners but capable of being comprised of various other structures known to connect to ends 21, 22 of a strap 20 together, may be removably connected to each other as shown in FIG. 7 to form a loop of the strap 20.

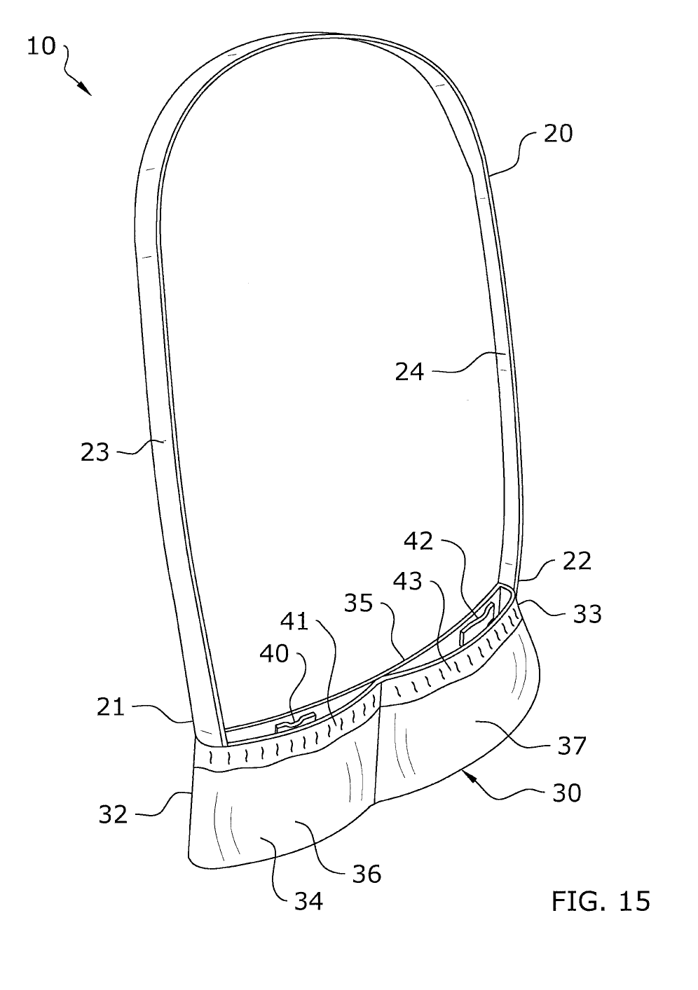

In some embodiments, the strap 20 may not form a loop with itself but instead may form a loop in combination with the retainer attachment 30. In such an embodiment, shown in FIG. 15, the strap 20 is secured to a retainer connector 38 extending from the retainer attachment 30. In such an embodiment, the strap 20 may include a first connector 26 which connects the first end 21 of the strap 20 to the strap 20 itself as shown in FIG. 15, with the second end 22 of the strap 20 being fixedly secured to the second end 33 of the retainer attachment 30. In other embodiments, the first connector 26 may comprise a tab of double-sided Velcro material (hook-and-loop fastener) which may be inserted between two portions of the strap 20 to secure the two portions of the strap 20 together. In such embodiments, the first connector 26 may be freely removable from the strap 20. In some embodiments, the length of the strap 20 may be variable in that the strap 20 may have its length increased or decreased. For example, a standard buckle may be utilized to allow the strap 20 to be comprised of a variable length configuration. Such a configuration is preferable to allow the strap 20 to tightly but comfortably fit around a range of differently-sized bodies.

Retainer Attachment

As shown throughout the figures, some of the disclosed embodiments utilize a retainer attachment 30 which acts to secure the tube 12 and bulb 13 therein in a secure fashion. The strap 20 allows the retainer attachment 30 to be secured at various portions of an individual's 14 body. The retainer attachment 30 may be fixedly or removably secured to the strap 20 in different embodiments.

In a first embodiment shown in FIG. 2, the retainer attachment 30 comprises a base 31 and a pair of pouches 36, 37 extending from the base 31. The base 31 will preferably comprise a soft material which acts as a cushion between the retainer connector 38 and the body. In such an embodiment, the base 31 comprises a first end 32, a second end 33, an outer surface 34, and an inner surface 35. The first end 32, the second end 33, or both ends 32, 33 of the base 31 may be removably or fixedly secured to the outer surface 23 of the strap 20. For example, in the embodiment shown in FIG. 3, the first end 32 of the base 31 is fixedly secured to the strap 20 while the second end 33 of the base 31 is removably secured to the strap 20 via a retainer connector 38, such as hook-and-loop fasteners. The base 31 preferably comprises a soft, cloth-like material to decrease discomfort to the individual 14 wearing it.

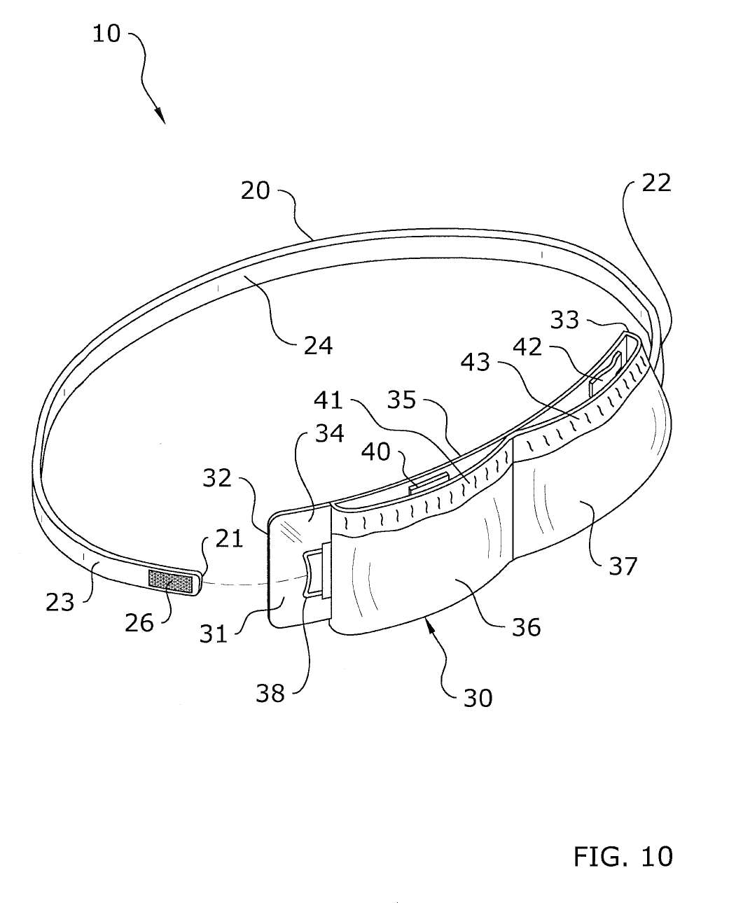

In the embodiment shown in FIGS. 10-13, the retainer connector 38 comprises a ring structure, with the strap 20 wrapping around the retainer connector 38 before the strap 20 connects to itself. In the embodiment shown in FIG. 15, it is shown that the base 31 is optional; with the strap 20 being connected directly to the pouches 36, 37. In such an embodiment, the retainer connector 38 extends from the pouches 36, 37 and comprises a ring structure through which the strap 20 is extended before connecting to itself. It should be appreciated that various other types of retainer connectors 38 may be utilized so long as the strap 20 is secured to retainer attachment 30 at either a base 31 or the pouches 36, 37.

Pouches and Tube Retainers

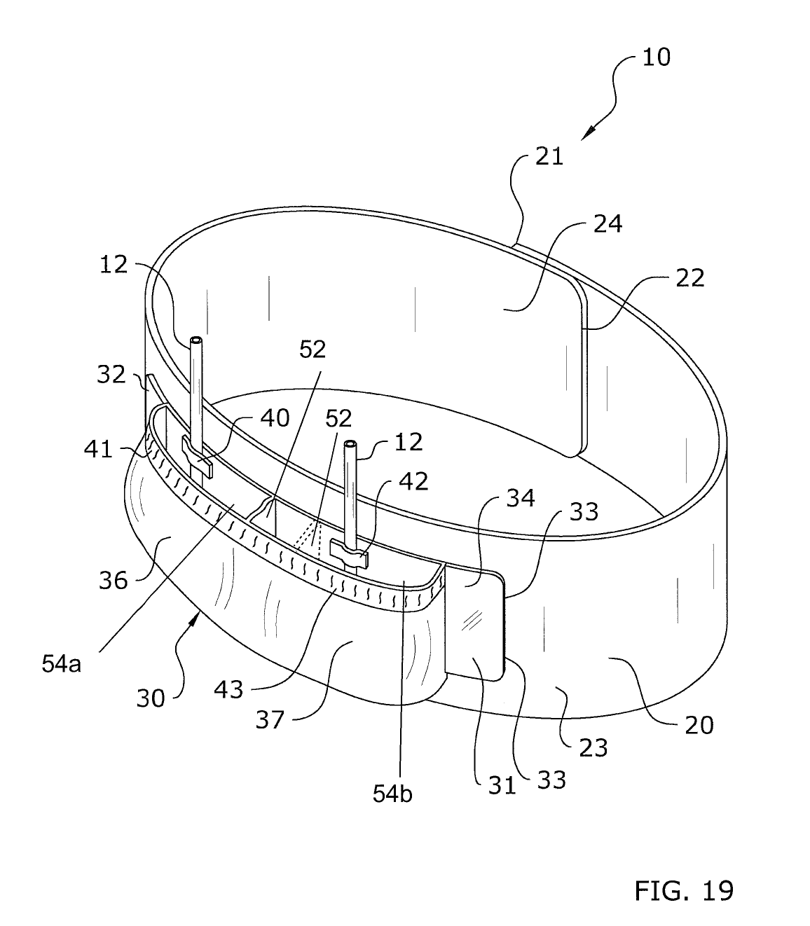

As shown throughout the figures, the retainer attachment 30 may include one or more pouches 36, 37 adapted to firmly and securely secure both the tube 12 and bulb 13 such that they do not move around or become compromised with movement of the individual 14. In the embodiment shown in the figures, a first pouch 36 and a second pouch 37 are utilized. However, it should be appreciated that more pouches 36, 37 may be utilized in some embodiments. The pouches 36, 37 may be divided by a sew-line as shown in FIG. 1-11 (i.e. a singular pouch divided into two by sewing across a central area) or may be divided by a piece of cloth or other such divider as shown in FIG. 19. The use of a divider in the pouches 36, 37 is to distribute the contents, such that all the contents do not bunch up on one side.

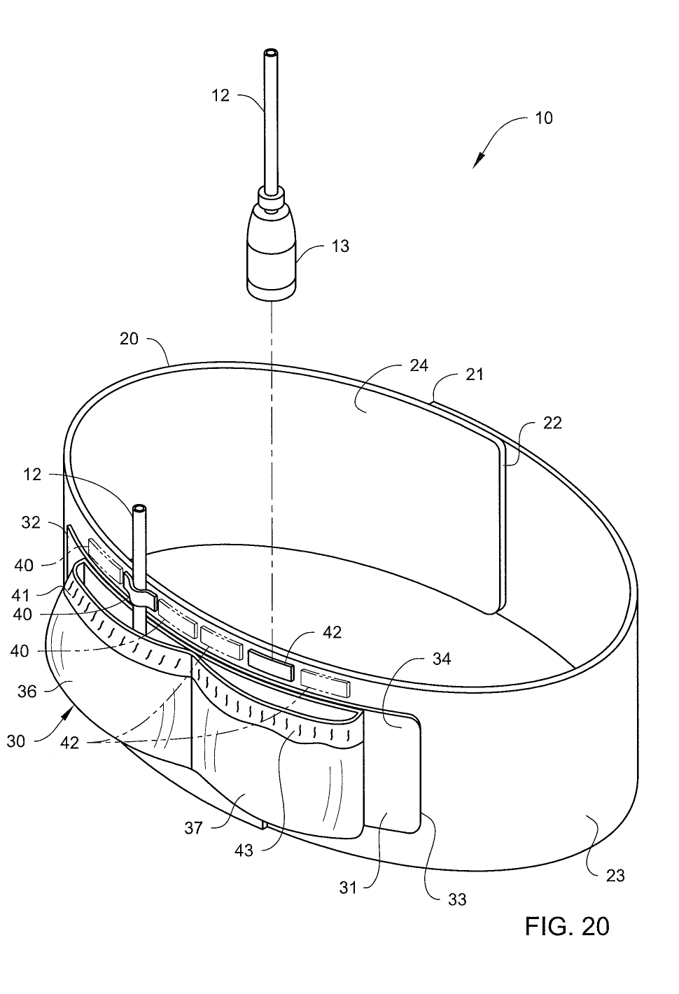

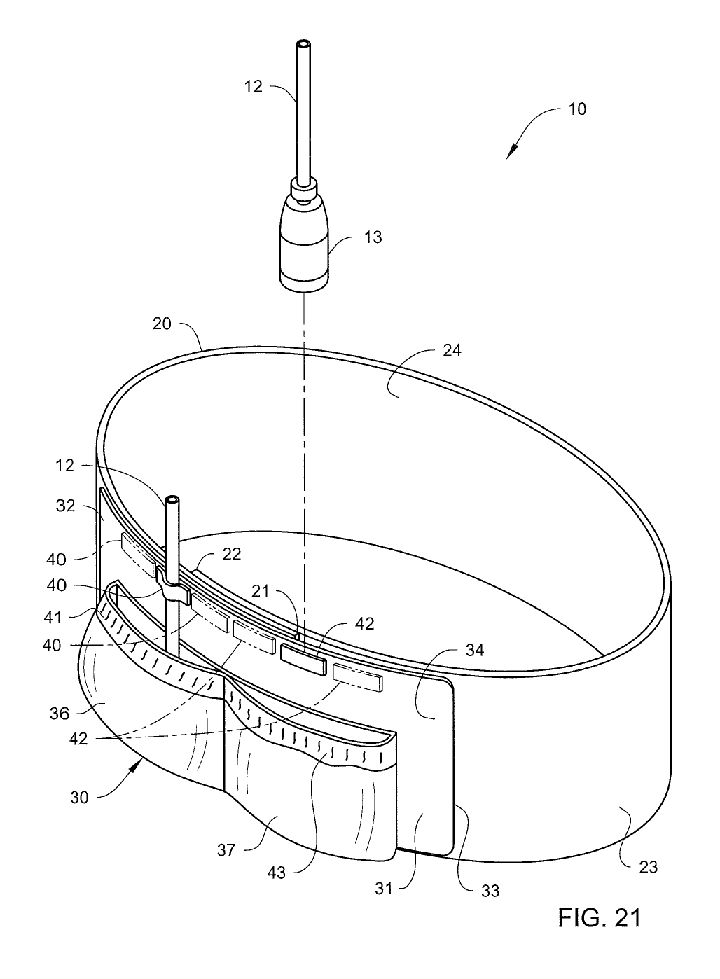

Referring to FIG. 19, in some embodiments, the retainer attachment 30 may include what can be referred to as a single pouch that is divided by a divider 52 into the two pouches 36, 37 (which may also be referred to as two sub-pouches 54a, 54b). For example, the pouch can be considered to be a single combined pouch, where the single pouch is sub-divided into the sub-pouches 54a, 54b by the divider 52 (e.g. the divider can be the piece of cloth or other divider as shown in FIG. 19 or a sew line as in FIGS. 1-11). Each of the sub-pouches 54a, 54b can include one or more tube retainers, like the tube retainers 40, 42, with the tube retainers located either at the opening of each sub-pouch 54a, 54b or located above the sub-pouches 54a, 54b as described below with respect to FIGS. 20 and 21.

Still referring to FIG. 19, the position of the divider 52 may also be adjustable in order to adjust the size of each of the sub-pouches 54a, 54b. For example, FIG. 19 illustrates the position of the divider 52 in dashed lines shifted to a different location whereby the sub-pouch 54a will be larger than the sub-pouch 54b. The divider 52 can be shifted in the other direction as well so that the sub-pouch 54b can be made larger than the sub-pouch 54a. The position of the divider 52 can be shifted and fixed in position using any suitable securement mechanism(s). For example, the ends of the divider 52 can include one or more hook and loop patches that can be secured to one or more hook and loop patches that are secured to the inside surfaces of the front and rear panels that form the pouch at various positions along the panels to provide for position adjustment of the divider 52. However, other securement mechanisms could be used as well such as snaps, buttons, and the like. In addition, two or more of the dividers 52 can be provided in the pouch in order to permit division of the pouch into more than two sub-pouches.

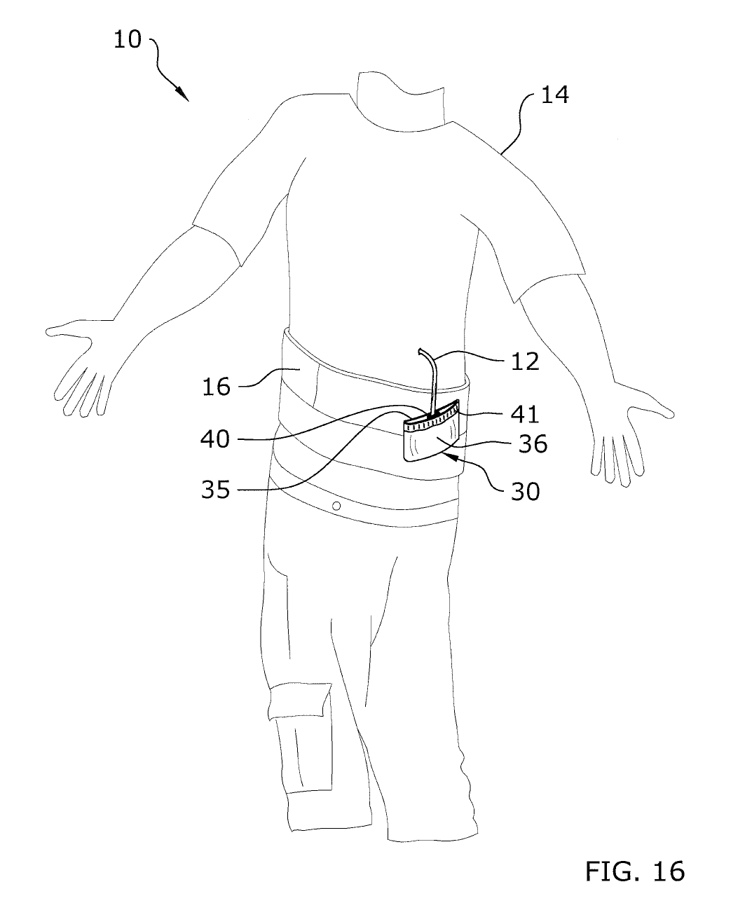

The number, orientation, size, and configuration of the pouches 36, 37 should not be construed as limited by the exemplary figures. FIGS. 1-11, 14, 15, and 19 show a first pouch 36 and a second pouch 37. FIGS. 12-13 and 16-18 show a configuration which only utilizes a first pouch 36. Further, in the embodiment shown in FIGS. 16-18, it can be seen that a singular pouch may be provided having a pouch retainer 39 such as a hook-and-loop fastener. The pouch retainer 39 may be utilized to removably secure the pouch 36 to various other items, such as to some types of clothing, to a bandage 16 as shown in FIGS. 16-18, or to the base 31 of a retainer attachment 30.

The pouches 36, 37 may comprise various materials and should not be construed as being limited to any particular material. However, a mesh-like material is preferable for embodiments of the pouches 36, 37 that may be used in the shower or the like so that fluids do not pool up within the pouches. Embodiments not intended for use in a shower may omit the mesh-like material.

As shown in the figures, the pouches 36, 37 may include elastic 41, 43 at their respective upper ends. Thus, the first pouch 36 includes a first elastic 41 at its upper end and the second pouch 37 includes a second elastic 43 at its upper end. The elastics 41, 43 aid with opening the pouches 36, 37 up to place items inside or remove items therefrom. Absent force, the elastics 41, 43 will revert to their resting positions.

Each of the pouches 36, 37 includes tube retainers 40, 42 adapted to secure the tube 12 and bulb 13 within the respective pouch 36, 37. FIG. 2 illustrates an embodiment in which the first pouch 36 includes a first tube retainer 40 and the second pouch 37 includes a second tube retainer 42. The structure, positioning, orientation, and size of the tube retainers 40, 42 may vary in different embodiments. The tube retainers 40, 42 may comprise straps which extend around the tube 12 before being secured via hook-and-loop fasteners or the like. The tube retainers 40, 42 may also comprise a flap of material, a clasp, a clip, or any other structure capable of securing the tube 12 within the pouch 36, 37.

In some embodiments, each pouch 36, 37 may include multiple tube retainers 40, 42 adapted to secure multiple tubes 12 and bulbs 13 within a single pouch 36, 37. Such an embodiment is shown in FIG. 7, in which the first pouch 36 includes a first retainer row 44 therein and the second pouch 37 includes a second retainer row 46 therein. In such an embodiment, the first retainer row 44 comprises a plurality of first tube retainers 40 and the second retainer row 46 comprises a plurality of second tube retainers 42.

FIG. 20 illustrates an embodiment that is similar to FIG. 2. However, in this embodiment, the tube retainer 40, 42 are located at a position above the pouches 36, 37 and therefore outside of the interior space of the pouches 36, 37. For example, the tube retainers 40, 42 are illustrated as being located on an outer surface 23 of the belt 20 above the openings to the pouches 36, 37. Additional tube retainers 40, 42 (shown in dashed lines) can also be located above the pouches 36, 37.

FIG. 21 illustrates another embodiment that is similar to FIG. 2. However, in this embodiment, the tube retainer 40, 42 are located on the base 31 of the retainer attachment 30 rather than on the belt 20 as in FIG. 20. In this embodiment, the tube retainers 40, 42 are again located at a position above the pouches 36, 37 and therefore outside of the interior space of the pouches 36, 37. The tube retainers 40, 42 are spaced higher above the pouches than in FIG. 20. Additional tube retainers 40, 42 (shown in dashed lines) can also be located above the pouches 36, 37. This embodiment also shows the location of the joint between the first end 21 and the second end 22 of the belt 20 at the front, with the retainer attachment 30 overlapping the first end 21, the second 22 and the joint between the two. In this embodiment, the end 33 (or both of the ends 32, 33) of the retainer attachment 30 can be disconnected from the belt 20 as described above for FIG. 3. The belt 20 can then be secured around the individual's waist or other body part. Thereafter, the end 33 can be re-secured to the belt 20 to help cinch the belt 20 tightly around the individual.

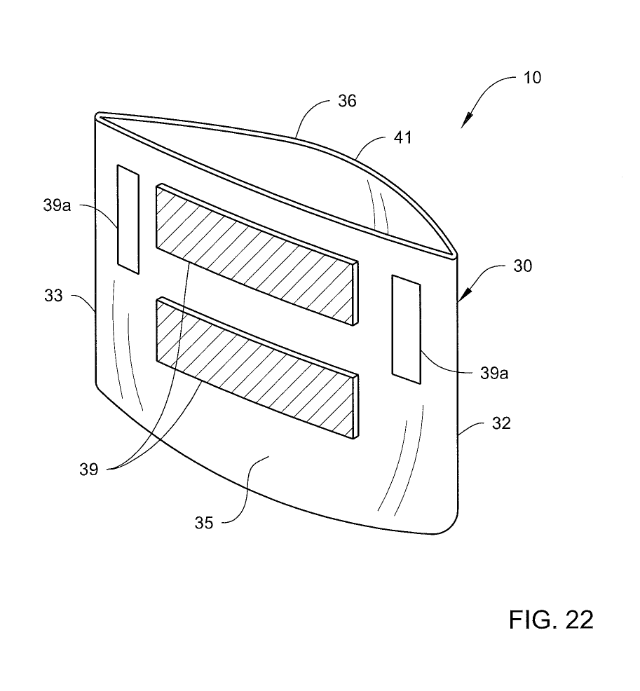

FIG. 22 shows an embodiment of a singular pouch that is similar to FIG. 18. In FIG. 22, a plurality of the pouch retainers 39, such as hook-and-loop fasteners, can be provided at different locations on the back side of the pouch 36 to help removably secure the pouch 36 to various other items, such as to some types of clothing, to a bandage 16 as shown in FIGS. 16-18, or to the base 31 of a retainer attachment 30. In addition, different pouch retainers 39a are illustrated schematically. The pouch retainers 39a can be fasteners with a construction different than hook-and-loop fasteners, that can be used separately from or together with the pouch retainers 39. For example, the pouch retainers 39a can be safety pins, snaps, buttons, or any other type of mechanical fastener.

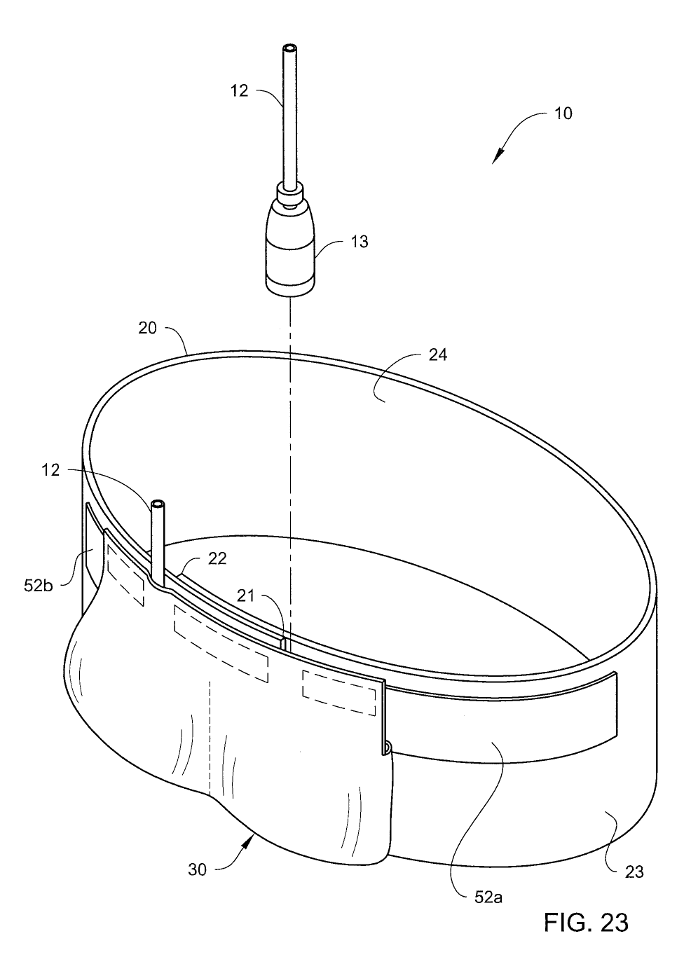

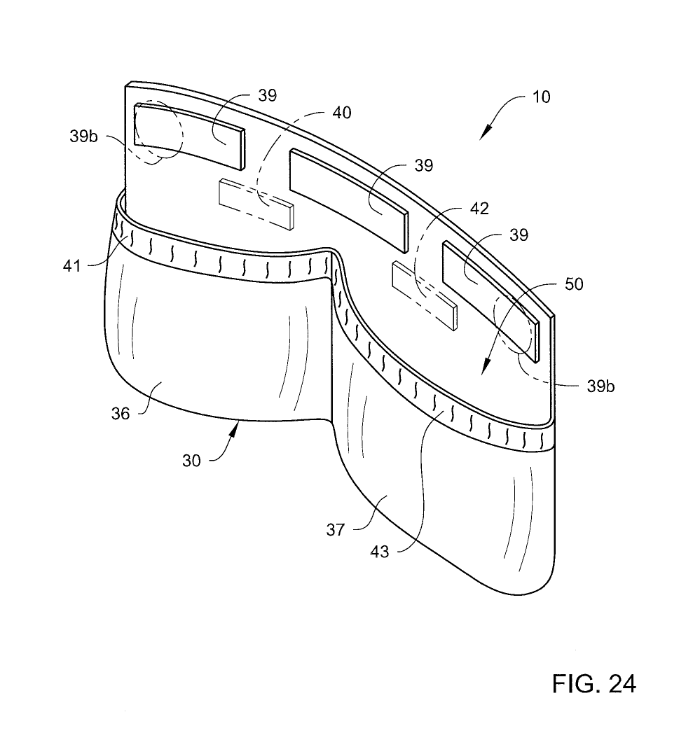

FIGS. 23 and 24 illustrate an embodiment of the drain tube holder system 10 where the pouch(es) faces the individual during use. In particular, the strap 20 is in the form of an abdominal binder with the first and second ends 21, 22 that overlap one another to secure the abdominal binder around the individual's waist with the outer surface 23 facing away from the individual and the inner surface 24 facing the individual. The retainer attachment 30 is detachably secured to the abdominal binder, with at least one pouch of the retainer attachment 30 facing inward toward the individual.

In the illustrated example, the retainer attachment includes a flap 50 and at least one of the pouches 36, for example both of the pouches 36, 37, can be formed adjacent a base end of the flap 50. As depicted in FIG. 23, the flap 50 may form a front surface of the pouch(es) 36, 37. Alternatively, the pouch(es) 36, 37 may be a separate structure that is attached to the flap 50. As shown in FIG. 24, a significant portion of the flap 50 extends above the top opening(s) of the pouch(es) 36, 37 to provide a surface on which one or more pouch retainers 39 can be disposed. The pouch retainer(s) 39 are configured to detachably secure the retainer attachment 30 to the abdominal binder. The pouch retainer(s) 39 can be any fastener mechanism suitable for detachably securing the retainer attachment 30 to the abdominal binder including, but not limited to, snaps, buttons, hook or loop pads of a hook and loop fastener system, and the like. Hook or loop pads are shown in FIG. 24. Fasteners 39b in the form of snaps or buttons are shown in dashed lines. The pouch retainer(s) 39, 39b detachably engage with corresponding fasteners on the abdominal binder. For example, in the case of the pouch retainer(s) 39 being a plurality of spaced hook pads, loop pads 52a, 52b can be provided on the outer surface 23. In some embodiments, the tube retainers 40, 42 (illustrated in broken lines) can optionally be provided above or even in the pouch(es) 36, 37.

In use, the retainer attachment 30 is oriented on the abdominal binder such that the pouch(es) 36, 37 face the individual wearing the abdominal binder, and the bulb 13 is disposed in the pouch so that the bulb 13 and much of the tube 12 are hidden behind the retainer attachment. The tube 12 may extend upwardly between the flap 50 and the outer surface 23 as depicted in FIG. 23 or the tube 12 may extend through a hole in the abdominal binder.

Operation

In use, it is preferable that the strap 20 be secured around the body of an individual 14 before inserting the tubes 12 and bulbs 13. The strap 20 may be secured around various portions of the body, such as the shoulder, the waist, the stomach/mid-section, the leg, the arm, or the like. The strap 20 may be adjusted to ensure a secure fit around the body before attempting to secure the tubes 12 and bulbs 13.

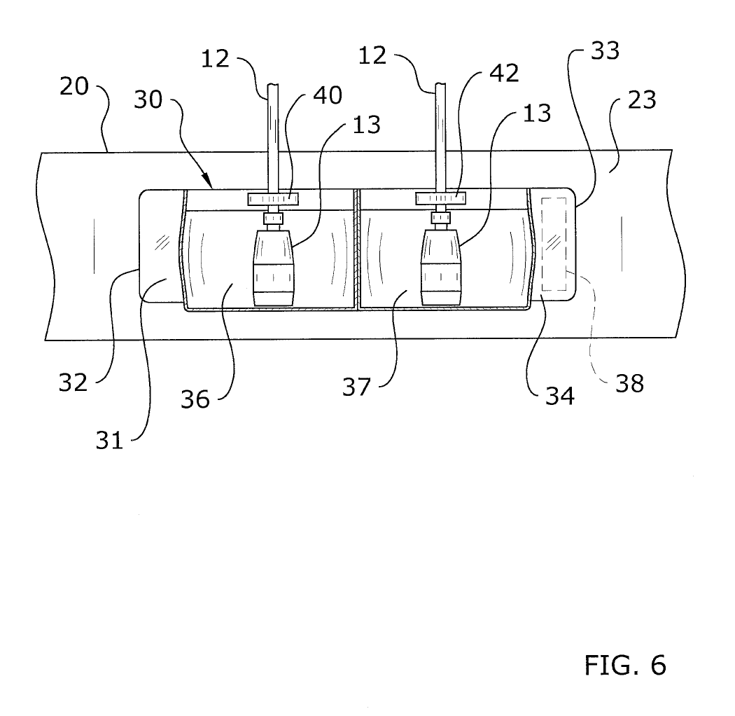

With the strap 20 secured around the body at the desired area, the drainage tubes 13 may be routed from their entrance/exit of the body to the drain tube holder systems described herein. Each drainage tube 13 is secured within a tube retainer 40, 42 of the pouches 36, 37 of the retainer attachment 30. The bulb 13 itself, which acts as a reservoir for any fluids drained through the tube 13, may be secured within the pouch 36, 37 itself as shown in FIG. 6. The individual 14 may then proceed with their daily tasks secure with the knowledge that the tubes 12 and bulbs 13 are firmly secured and thus are not likely to be moved around, disconnected, or otherwise compromised.

The examples disclosed in this application are to be considered in all respects as illustrative and not limitative. The scope of the invention is indicated by the appended claims rather than by the foregoing description; and all changes which come within the meaning and range of equivalency of the claims are intended to be embraced therein.

1. A drain tube holder system, comprising:
a strap including a first end and a second end, wherein said first end of said strap is removably connected to said second end of said strap;
a retainer attachment connected to said strap, wherein said retainer attachment includes a first pouch defining an interior space sufficient to retain a reservoir bulb therein;
the first pouch includes a first side, a second side opposite the first side, a lower end, and an upper end opposite the lower end, the upper end being open to allow a drain tube that is connected to a reservoir bulb within the first pouch to extend therethrough; and
at least one tube retainer associated with the first pouch, the at least one tube retainer is located on the strap or on the retainer attachment above the upper end and outside the interior space, and the at least one tube retainer is configured to secure a drain tube that extends through the upper end of the first pouch.

2. The drain tube holder system of claim 1, wherein the retainer attachment is disposed over the first end and the second end of the strap.

3. The drain tube holder system of claim 1, comprising a plurality of the tube retainers associated with the first pouch and located on the strap or on the retainer attachment above the upper end and outside the interior space.

4. The drain tube holder system of claim 1, wherein the retainer attachment includes a second pouch defining an interior space sufficient to retain a reservoir bulb therein;
the second pouch includes a first side, a second side opposite the first side, a lower end, and an upper end opposite the lower end thereof, the upper end of the second pouch being open to allow a drain tube that is connected to a reservoir bulb within the second pouch to extend therethrough; and
at least one tube retainer associated with the second pouch, the at least one tube retainer associated with the second pouch is located on the strap or on the retainer attachment above the upper end of the second pouch and outside the interior space of the second pouch, and the at least one tube retainer of the second pouch is configured to secure a drain tube that extends through the upper end of the second pouch.

5. The drain tube holder system of claim 4, comprising a plurality of the tube retainers associated with the second pouch and located on the strap or on the retainer attachment above the upper end of the second pouch and outside the interior space of the second pouch.

6. A drain tube holder system, comprising:
a strap including a first end and a second end, wherein said first end of said strap is removably connected to said second end of said strap;
a retainer attachment connected to said strap, wherein said retainer attachment includes a first pouch defining an interior space sufficient to retain a reservoir bulb therein;
the first pouch includes a first side, a second side opposite the first side, a lower end, and an upper end opposite the lower end, the upper end being open to allow a drain tube that is connected to a reservoir bulb within the first pouch to extend therethrough;
at least one tube retainer associated with the first pouch, the at least one tube retainer is configured to secure a drain tube that extends through the upper end of the first pouch;
a divider within the interior space of the first pouch, the divider is removably secured to the first pouch so that the position of the divider within the interior space of the first pouch is adjustable.

7. The drain tube holder system of claim 6, wherein the at least one tube retainer is located within the first pouch or located above the upper end and outside the interior space.

8. A drain tube holder system, comprising:
a reservoir bulb holding pouch having an interior wall and an exterior wall secured to one another along a first side, a second side, and a lower end to define an interior space sufficient in size to completely retain a reservoir bulb therein;
an upper end of the interior wall and the exterior wall being open to allow a drain tube that is connected to a reservoir bulb disposed completely within the interior space to extend therethrough;
the interior wall includes a first surface and a second surface, and the exterior wall includes a third surface and a fourth surface;
the second surface faces the third surface;
at least one tube retainer on the second surface of the interior wall at the upper end thereof, the at least one tube retainer is configured to secure a drain tube that extends through the upper end;
at least one pouch retainer fixed to the first surface of the interior wall, the at least one pouch retainer is configured to removably secure the reservoir bulb holding pouch to another item; and
elastic on the exterior wall at the upper end thereof.

9. The drain tube holder system of claim 8, comprising a plurality of the pouch retainers fixed to the first surface of the interior wall, each one of the pouch retainers is configured to removably secure the reservoir bulb holding pouch to the another item.

10. A retainer attachment of a drain tube holder system, comprising:
a flap having a base end and an upper surface facing in a first direction;
at least one reservoir bulb holding pouch adjacent to the base end of the flap, the at least one reservoir bulb holding pouch defining an interior space sufficient in size to retain a reservoir bulb therein;
the at least one reservoir bulb holding pouch includes a first side, a second side opposite the first side, a lower end, and an upper end opposite the lower end, the upper end being open to allow a drain tube that is connected to the reservoir bulb within the interior space to extend therethrough, and the upper end faces toward the upper surface; and
the upper surface of the flap extends above the upper end of the at least one reservoir bulb holding pouch, and at least one pouch retainer is disposed on the first surface, the at least one pouch retainer is configured to detachably secure the retainer attachment to an abdominal binder secured around a patient so that the at least one reservoir bulb holding pouch faces in the first direction.

11. The retainer attachment of a drain tube holder system of claim 10, comprising a plurality of the reservoir bulb holding pouches adjacent to the base end of the flap.
